(19) United States
(12) Patent Application Publication
(10) Pub. No.: US 2026/0217857 A1
(43) Pub. Date: Jul. 30, 2026

(54) MULTI-FUNCTIONAL PROTEIN COMPOSITIONS AND METHODS OF MAKING AND USE THEREOF

(71) Applicant: The Texas A&M University System, College Station, TX (US)

(72) Inventors: Qing Sun, College Station, TX (US); Baizhen Gao, College Station, TX (US)

(21) Appl. No.: 19/466,206

(22) Filed: Jan. 30, 2026

Related U.S. Application Data
(60) Provisional application No. 63/751,552, filed on Jan. 30, 2025.

(51) Int. Cl. C07K 16/30; A61K 38/00; A61K 45/06; A61K 47/42; A61P 35/00; C07K 16/28; C07K 16/32; G01N 33/575; G01N 33/68

(52) U.S. Cl. CPC C07K 16/30; A61K 38/00; A61K 45/06; A61K 47/42; A61P 35/00; C07K 16/2809; C07K 16/2863; C07K 16/32; G01N 33/575; G01N 33/68; C07K 2318/20

(57) ABSTRACT

Provided here are multi-functional compositions containing an elastin-like-polypeptide (ELP) scaffold containing two or more antigen-binding components or biological response modifiers. These multi-functional compositions can be used for detection or therapeutic treatment of diseases. Provided here are multi-antigen-binding compositions, each containing two or more antigen-binding components coupled by an elastin-like-polypeptide (ELP) scaffold. These multi-antigen-binding compositions can be used for detection or for therapeutic treatment of a disease.

### CROSS-REFERENCE TO RELATED APPLICATIONS

This application claims the benefit of and priority to U.S. Provisional Application No. 63/751,552, filed Jan. 30, 2025, which is incorporated by reference herein in its entirety.

### GOVERNMENT SUPPORT

This invention was made with government support under grant numbers 2221892 from the National Science Foundation and 2132156 awarded by NSF Emerging Frontiers in Research and Innovation. The government has certain rights in the invention.

### TECHNICAL FIELD

The disclosure relates to multi-functional protein compositions assembled via highly specific protein-protein interactions.

### BACKGROUND

Traditional monoclonal antibody therapeutics have been successful as treatment options for various conditions; however, their efficacy can be constrained by their specificity for a single target, while diseases often stem from diverse factors and mediators. Multi-functional antibodies are a special class of antibodies that simultaneously bind to multiple targets. This property offers numerous advantages for the multi-functional antibodies over the traditional monoclonal antibodies. First, they have higher binding specificity towards the target because they can engage multiple different epitopes simultaneously. Second, the ability to bind two or more different antigens enables the blocking of multiple independent or overlapping pathways in pathogenesis for better therapeutic efficacy. Third, multi-specific antibodies can redirect immune cells, such as T cells, to tumor cells and enhance the therapeutic efficacy. And lastly, producing one antibody that can target multiple different epitopes simultaneously is more cost effective than producing multiple traditional monoclonal antibodies for combination therapy.

For monoclonal antibodies, all FDA approved monoclonal antibodies are IgGs. Each IgG consists of two identical fragment antigen-binding (Fab) domains that are responsible for recognizing and binding with the targets, and one fragment crystallizable (Fc) region that prolongs IgG serum half-life through neonatal crystallizable fragment receptor (FcRn) mediated recycling and induces immune responses including antibody dependent cellular cytotoxicity (ADCC).

Compared to monoclonal antibodies, multi-functional antibodies can be produced as either IgG-like or non-IgG like. IgG-like multi-functional antibodies have a similar structure to monoclonal IgGs except for possessing two distinct Fabs that can bind to different targets. The presence of Fc extends the in vivo half-life and makes IgG-like multi-functional antibodies generally bigger than non-IgG like antibodies. IgG-like multi-functional antibodies are often made by hybridization between different heavy chains or light chains, but this asymmetric structure can lead to high probability of mispairing. Various strategies have been developed to promote the correct pairing during production and purification including knobs-into-holes, mutations encouraging salt bridge formation between heterogeneous heavy chains, and mutations changing the binding affinity towards protein A columns. Although promising, these approaches require rounds of sophisticated engineering and usually lead to lower yield and high cost.

Non-IgG-like multi-functional antibodies are often made by fusing different antibody fragments such as a Fab, a single-chain variable fragment (scFv), and a nanobody. Compared to IgG-like multi-functional antibodies, they are easier to produce and have low immunogenicity. Genetic fusion of different antibody fragments also has its own challenges. Fusing too many fragments can cause instability and aggregation problems, and different antibody fragments can only be fused in N to C or C to N terminal orientations, limiting the number and relative geometries of different fragments that can be combined.

Because of the challenges and limitations of producing multi-functional antibodies, different approaches have been developed to assemble multi-functional antibodies post-translationally. Compared to IgG-like or genetic fusion of non-IgG-like multi-functional antibodies, post-translational assembly offers more flexibility in the structure and orientation of the multi-functional antibody. The methods that have been developed to assemble multi-functional antibodies include chemical conjugation, nucleic acids mediation, and protein-protein interaction.

Chemical conjugation has enabled many of the first-generation multi-functional antibodies. The most commonly used conjugation methods include lysine conjugation, cysteine conjugation, disulfide bond bridging, and conjugation using unnatural amino acids. Lysine conjugation is often used to assemble two full-length IgGs. Recently, a modified peptide Fc-III peptide has been used to site selectively bind to Lys248, a “consensus” site of the IgG Fc, through acetylation. The peptide further enabled derivatization by DBCO-PEG4-TAMRA (DBCO, dibenzocyclooctyne; PEG, poly(ethylene glycol); TAMRA, carboxytetramethylrhodamine) through a strain-promoted azide-alkyne cycloaddition (SPAAC) reaction for multi-functional antibody assembly to achieve antibody lipidation and bispecificity. An assembled Trastuzumab and OKT3 were able to induce around 70% cytotoxicity, demonstrating the effectiveness of this approach. Although early generations of multi-functional antibodies produced by chemical conjugation led to heterogeneous products that require additional purification steps, it provided an approach to bind to different targets using formats that are not feasible with molecular engineering methods. Site-specific conjugation methods significantly improved the conjugation selectivity, but overall yield remains challenging.

Besides direct linkage between different antibodies, nanoparticles have also been used as carriers for the assembly of multi-functional antibodies for both cancer cell detection and therapeutic applications. Besides organic materials, inorganic materials such as Fe₃O₄ have also been used as the nanocarrier. Adoption of nanoparticles as carriers provided the capability of assembling the antibody with different ratios and increased the avidity of each assembled product, although the increased size may prevent it from reaching certain targets. The ability to assemble and dissociate antibodies in vivo further enabled more effective antibody administration strategies without causing prolonged toxic side effects.

Besides chemical modification, oligonucleotides have emerged as versatile building blocks to assemble various structures including nanoparticle arrays and protein nanostructures. By conjugating the oligonucleotides on the proteins, the assembly process is highly selectivity because of the remarkable specificity of Watson-Crick base pairing. The exceptional specificity, coupled with the freedom to design custom oligonucleotide sequences, offers unparalleled control over the spatial arrangement of the assembled products compared to the conjugation achieved by small chemical molecules. This unique property has enabled the successful assembly of multi-functional antibodies.

Although multi-functional antibodies were successfully assembled using oligonucleotides, it required an additional step to incorporate unnatural amino acids. Recently, a method was developed to assemble any proteins and does not require mutations or specific amino acids such as cysteine; this method included two distinct 20 base nucleic acid sequences strain-promoted azide-alkyne cycloaddition through strain-promoted azide-alkyne cycloaddition (SPAAC) reaction. The conjugated antibodies were then mixed to from antibody dimer, trimer, and pentamer. The antigen binding and checkpoint inhibitor activity retained after assembly, and the assembled products exhibited minimal degradation. In contrast to conjugation methods that uses small chemical molecules, which typically allow the assembly of only two different antibodies in many cases, oligonucleotide-mediated approach has been used to the assemble up to 5 antibodies. This method offers the advantage of allowing precise control over the spatial orientation and composition of the assembled products through the design of oligonucleotide sequences. However, despite significant progress in making protein-nucleotide complexes, the conjugation of oligonucleotides onto antibodies remains a potential limiting step for a wider adoption of the method.

Assembly of multi-functional antibodies via protein-protein interactions often avoids the additional conjugation steps needed to add the chemicals or oligonucleotides in both chemical conjugation and nucleic acids mediated assembly. With this advantage, several protein-protein interactions have been developed to assemble multi-functional antibodies, such as the SpyTag-SpyCatcher interaction. Originated from the bacterium Streptococcus pyogenes, SpyTag and SpyCatcher spontaneously and irreversibly form a covalent bond when mixed. Following the development of SpyTag-SpyCatcher, an orthogonal pair SnoopTag-SnoopCatcher has also been engineered that works similarly. Sortase mediated ligation (SML) is another commonly used method to assemble two proteins. Sortase A recognizes short tags LPXTG and GGG, cleaves between the threonine and the glycine residues, forms a thioester acyl-enzyme intermediate, and catalyzes the covalent linkage of the carboxyl group of the threonine to a nucleophilic amino group of a N-terminal glycine. This reaction is highly specific and readily occurs at room temperature. The small tags with less than 5 amino acids also make SML an attractive way to assemble multi-functional antibodies. Full-length IgGs and scFvs have been successfully assembled into multi-functional antibodies using SML and demonstrated neutralization potency against H1 and H3 HA proteins for influenza. Using similar approach, Fabs have been assembled to inhibit CD20-positive tumor growth in mouse xenograft model by targeting CD3 and CD20 simultaneously. However, in the case of SML, a chemical conjugation step is often still required to assemble the different building blocks.

Split-inteins have also been used to conjugate proteins in vitro. Compared to other assembly methods using protein-protein interactions, one benefit of using split inteins is that this process does not add additional molecular weight to the assembled product, and thus has the minimal influence on the antibody structure. Besides the common bioconjugation methods mentioned above, other protein-protein interactions such as L27 and ubiquitin have also been utilized for assembling multi-functional antibodies. Compared to chemical conjugation and oligonucleotide assisted assembly, protein-protein interactions often allow the antibodies to be assembled directly after synthesis without additional modifications except for SML. However, the addition of protein interaction pairs such as SpyTag/SpyCatcher also increases the antibody size more significantly than chemical conjugation or oligonucleotides. The greater size can be beneficial by helping extend the in vivo half-life, but also reduces their diffusivity in solid tumors.

### SUMMARY

Applicant recognized the need for multifunctional compositions and methods of making them that address these shortcomings of the art and provide other additional or alternative advantages. Provided here are multi-functional compositions containing an elastin-like-polypeptide (ELP) scaffold containing two or more antigen-binding components or biological response modifiers. In some embodiments, the biological response modifier is an immunomodulatory agent. These multi-functional compositions can be used for affecting a change to an immune response, for detection of a disease, or for therapeutic treatment of several diseases. These diseases can be an infectious disease, an autoimmune disease, an inflammatory disease, a metabolic disorder, a cardiovascular disease, a fibrotic disease, a neurological disorder, or a cancer.

Embodiments of a multi-functional composition can include an elastin-like-polypeptide (ELP) scaffold containing two or more antigen-binding components or biological response modifiers and two or more specific orthogonal protein interaction pairs. In some embodiments, specific orthogonal protein interaction pairs are two or more of SpyTag/SpyCatcher, SpyTag/KTag, Isopeptag/pilin-C, SnoopTag/SnoopCatcher, SpyTag002/SpyCatcher002, cohesin/dockerin, DogTag/DogCatcher, and SdyTag/SdyCatcher. In some embodiments, the multi-functional composition is used in detection of a biomarker indicative of an infectious disease, an autoimmune disease, an inflammatory disease, a metabolic disorder, a cardiovascular disease, a fibrotic disease, a neurological disorder, or a cancer. In some embodiments, the multi-functional composition includes a reporter. The reporter can be a fluorescent reporter, a bioluminescent reporter, an enzymatic reporter, or an affinity tag. In some embodiments, the biomarker is one or more of HER2 (Human Epidermal Growth Factor Receptor 2), KRAS (Kirsten Rat Sarcoma Viral Oncogene Homolog), EGFR (Epidermal Growth Factor Receptor), BRAF (B-Raf Proto-Oncogene, Serine/Threonine Kinase), BRCA1/BRCA2 (Breast Cancer Genes 1 or 2), prostate-specific antigen, carcinoembryonic antigen, and alpha-fetoprotein. In some embodiments, the biomarker is one or more of C-reactive protein, procalcitonin, serum amyloid A, cytokines (IL-1, IL-6, TNF-α, calprotectin, ferritin, fibrinogen, cardiac troponins, BNP/NT-proBNP, and Lipoprotein(a), CK-MB and microRNAs.

In some embodiments, the multi-functional composition is used in treatment of an infectious disease, an autoimmune disease, an inflammatory disease, a metabolic disorder, a cardiovascular disease, a fibrotic disease, a neurological disorder, or a cancer. In certain embodiments for the treatment of a cancer, a first antigen-binding component and a second antigen-binding component are directed to two of HER2, KRAS, EGFR, BRAF, and BRCA1/BRCA2. In some embodiments, the multi-functional composition contains one tumor-specific antigen-binding component and one T cell surface protein-binding fragment. In certain embodiments, the T cell receptor-binding fragment is directed to CD3 or is an anti-CD3 agent.

Embodiments include multi-antigen-binding compositions. In certain embodiments, the composition includes two or more antigen-binding components coupled to an elastin-like-polypeptide (ELP) scaffold utilizing two or more specific orthogonal protein interaction pairs. In certain embodiments, the specific orthogonal protein interaction pairs are two or more of SpyTag/SpyCatcher, SpyTag/KTag, Isopeptag/pilin-C, SnoopTag/SnoopCatcher, SpyTag002/SpyCatcher002, cohesin/dockerin, DogTag/DogCatcher, and SdyTag/SdyCatcher.

In certain embodiments, the multi-antigen-binding composition is used for detection of a biomarker indicative of an infectious disease, an autoimmune disease, an inflammatory disease, a metabolic disorder, a cardiovascular disease, a fibrotic disease, a neurological disorder, or a cancer. In certain embodiments, the biomarker is one or more of HER2, KRAS, EGFR, BRAF, BRCA1/BRCA2, prostate-specific antigen, carcinoembryonic antigen, and alpha-fetoprotein. In certain embodiments, a first antigen-binding component and a second antigen-binding component of the multi-antigen-binding composition are directed to detection of two of HER2, KRAS, EGFR, BRAF, and BRCA1/BRCA2. In some embodiments, the biomarker is one or more of C-reactive protein, procalcitonin, serum amyloid A, cytokines (IL-1, IL-6, TNF-α, calprotectin, ferritin, fibrinogen, cardiac troponins, BNP/NT-proBNP, and Lipoprotein(a), CK-MB and microRNAs.

In certain embodiments of the multi-antigen-binding composition, a first antigen-binding component binds to a tumor-specific antigen and a second antigen-binding component binds to T cell surface protein. In certain embodiments, the second antigen-binding component is anti-CD3. In certain embodiments of the multi-antigen-binding composition, a first antigen-binding component is anti-EGFR and a second antigen-binding component is anti-HER2. In certain embodiments of the multi-antigen-binding composition, a first antigen-binding component is anti-EGFR, a second antigen-binding component is anti-HER2, and a third antigen-binding component is anti-CD3.

Embodiments include a modular post-translational platform with highly specific protein-protein interactions for multi-functional antibody assembly and Elastin-like-polypeptide (ELP). Embodiments include methods of making these multi-functional antibodies that include the SpyCatcher002, SnoopCatcher, and dockerin fused with biocompatible ELP.

### BRIEF DESCRIPTION OF THE DRAWINGS

The patent or application file contains at least one drawing/photograph executed in color. Copies of this patent or patent application publication with color drawing(s)/photograph(s) will be provided by the Office upon request and payment of the necessary fees.

The present disclosure can be better understood by referring to the following figures. These drawings illustrate the principles of the disclosure and no limitation of the scope of the disclosure is thereby intended.

FIG. 1 is a schematic illustration of post-translational assembly of multi-functional antibody, according to an embodiment.

FIG. 2A demonstrates the purification of ELP scaffold (55.7 kDa) using 1M (NH₄)₂SO through two cycles of thermal precipitation and cold re-solubilization. With respect to the samples loaded on the lanes on the image of the Western Blot analysis presented in FIG. 2A, Lane 1 is of the total cell lysate; Lane 2 is of the supernatant after the first thermal precipitation; Lane 3 is of the resolubilized pellet after the first thermal precipitation; Lane 4 is of the supernatant containing purified ELP scaffold after the first cold spin; Lane 5 is of the resolubilized pellet after the first cold spin; Lane 6 is of the supernatant after the second thermal precipitation; Lane 7 is of the resolubilized pellet after the second thermal precipitation; Lane 8 is of the final purified ELP scaffold product after the second cold spin; and Lane 9 is of the resolubilized pellet after the second cold spin.

FIG. 2B demonstrates the assembly and purification of the antibodies. With respect to the samples loaded on the lanes on the image of the Western Blot analysis presented in FIG. 2B, Lane 1 is of the purified ELP scaffold; Lane 2 is of the purified 7D12-SpyT (20.4 kDa); Lane 3 is of the purified EgB4-SnoT (20.4 kDa); Lane 4 is of the purified GFP-coh (48.7 kDa); Lane 5-7 are samples after incubating 7D12-SpyT, EgB4-SnoT, and both 7D12 and EgB4 with ELP scaffold and GFP-coh; Lanes 8-10 are of the purified assembled antibody with 7D12 only (76.1 kDa), EgB4 only (76.1 kDa), and with both 7D12 and EgB4 (96.5 kDa).

FIG. 3A is a schematic illustration of the antibody binding assay. FIGS. 3B and 3D are the phase contrast and fluorescence images of MDA-MB-231 and A549 cells, respectively, incubated with 5 μM of different antibodies using GFP as the detection output. FIGS. 3C and 3E are graphical representations of the fluorescence intensity quantification of MDA-MB-231 and A549 cells, respectively, incubated with different antibodies.

FIG. 4A is a schematic illustration of using the assembled antibody to inhibit cancer cell growth and FIG. 4B presents the SDS gel analysis of antibody assembly for cancer cell growth inhibition. With respect to the samples loaded on the lanes on the image of the Western Blot analysis presented in FIG. 4B, Lane 1 is of the ELP scaffold (55.7 kDa); Lane 2 is of the 7D12− SpyT (20.4 kDa); Lane 3 is of the (Z_{HER2:4})₂-SnoT (19.6 kDa); Lane 4 is of the assembled 7D12 (76.1 kDa); Lane 5 is of the assembled (Z_{HER2:4})₂ (75.3 kDa); Lane 6 is of the assembled 7D12+ (Z_{HER2:4})₂ (95.7 kDa). FIG. 4C is a graphical representation of relative growth inhibition measured by MTT assay at a concentration of 5 μM for each assembled antibody. FIG. 4D is a schematic illustration of using the assembled antibody to direct T-cell for cancer cell cytolysis. FIG. 4E presents the SDS gel analysis of antibody assembly for T cell mediated cytolysis; the lower band on SDS indicates CD3-Coh at 36.8 kDa. FIG. 4F presents the T cell mediated cytolysis measured by LDH assay at a concentration of 1 μM for each assembled antibody.

### DETAILED DESCRIPTION

In the following description, numerous details are set forth in order to provide a thorough understanding of the various embodiments. In other instances, well-known processes, devices, and systems may not been described in particular detail in order not to unnecessarily obscure the various embodiments. Additionally, illustrations of the various embodiments may omit certain features or details in order to not obscure the various embodiments.

The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Provided here are multi-functional compositions containing an elastin-like-polypeptide (ELP) scaffold with two or more antigen-binding components or biological response modifiers. In some embodiments, the biological response modifiers act outside the direct immune system and modulate metabolic, inflammatory, and cellular processes to manage diseases. In some embodiments, the biological response modifier is an immunomodulatory agent. These multi-functional compositions can be used for detection or for therapeutic treatment of a disease or a disorder. These diseases can be an infectious disease, an autoimmune disease, an inflammatory disease, a metabolic disorder, a cardiovascular disease, a fibrotic disease, a neurological disorder, or a cancer. An antigen-binding component can be a full-size antibody, an antigen binding fragment, an engineered antibody fragment, a nanobody, or an affibody.

Embodiments of a multi-functional composition can include an ELP scaffold containing two or more antigen-binding components or biological response modifiers and two or more specific orthogonal protein interaction pairs. In some embodiments, specific orthogonal protein interaction pairs are two or more of SpyTag/SpyCatcher, SpyTag/KTag, Isopeptag/pilin-C, SnoopTag/SnoopCatcher, SpyTag002/SpyCatcher002, cohesin/dockerin, DogTag/DogCatcher, and SdyTag/SdyCatcher. In some embodiments, the multi-functional composition is used in detection of a biomarker indicative of an infectious disease, an autoimmune disease, an inflammatory disease, a metabolic disorder, a cardiovascular disease, a fibrotic disease, a neurological disorder, or a cancer. In some embodiments, the multi-functional composition includes a reporter. The reporter can be a fluorescent reporter, a bioluminescent reporter, an enzymatic reporter, or an affinity tag. These reporters can be chemical or biological entities. Examples of fluorescent reporters, include green fluorescent protein (GFP), enhanced GFP (eGFP), yellow fluorescent protein (YFP), red fluorescent protein (RFP), and tdTomato. Examples of bioluminescent reporters include Firefly Luciferase (FLuc), Renilla Luciferase (RLuc), and NanoLuc. Examples of enzymatic reporters include beta-galactosidase (β-gal) or alkaline phosphatase. In some embodiments, the biomarker is one or more of HER2, KRAS, EGFR, BRAF, BRCA1/BRCA2, prostate-specific antigen, carcinoembryonic antigen, and alpha-fetoprotein. In some embodiments, the biomarker is one or more of C-reactive protein, procalcitonin, serum amyloid A, cytokines (IL-1, IL-6, TNF-α, calprotectin, ferritin, fibrinogen, cardiac troponins, BNP/NT-proBNP, and Lipoprotein(a), CK-MB and microRNAs.

In some embodiments, the multi-functional composition is used in treatment of an infectious disease, an autoimmune disease, an inflammatory disease, a metabolic disorder, a cardiovascular disease, a fibrotic disease, a neurological disorder, or a cancer. In certain embodiments for the treatment of a cancer, a first antigen-binding component and a second antigen-binding component are directed to two of HER2, KRAS, EGFR, BRAF, and BRCA1/BRCA2. In some embodiments, the multi-functional composition contains one tumor-specific antigen-binding component and one T cell surface protein-binding fragment. In certain embodiments, the T cell receptor-binding fragment is directed to CD3 or is an anti-CD3 agent.

Multi-functional antibodies, capable of simultaneously engaging multiple targets, are a unique class of antibodies that has sparked growing interest. Current approaches for making multi-functional antibodies, including chemical conjugation or genetic modifications, suffer from low product yield, complex structure design, and complicated manufacturing processes.

Multi-functional antibodies exhibit enhanced binding affinity and specificity, which lower the risk for on-target off-tumor specificities. Multi-functional antibodies can direct specific immune cells to tumors. The Federal Drug Agency (FDA) and European Medicine Agency (EMA) have granted approval for blinatumomab, a bispecific T-cell engager that simultaneously attaches to T cells and cancer cells, enabling T cells to find and destroy the cancer cell by bringing them close together. Blinatumomab has shown to be more effective than chemotherapy in treating children and young adults with B-ALL that has come back after initial treatment. By targeting multiple antigens on a single target cell, multi-functional antibodies may prevent antigen-negative relapses. This is achieved by exerting therapeutic pressure through either sequential or simultaneous targeting, which helps inhibit the development of negative variants. Although promising, the intricate structures of multi-functional antibodies pose a significant challenge in their production. Current strategies, including IgG-like or non-IgG-like multi-functional antibody manufacturing, using genetic engineering and chemical modification, suffer from reduced yield, increased purification complexity, and restricted spatial geometric arrangements of domains.

Provided here are also multi-antigen-binding compositions, each containing two or more antigen-binding components coupled by an ELP scaffold. Certain embodiments of these multi-antigen-binding compositions can be used for detection or for therapeutic treatment of a disease. These diseases can be an infectious disease, an autoimmune disease, an inflammatory disease, a metabolic disorder, a cardiovascular disease, a fibrotic disease, a neurological disorder, or a cancer.

Embodiments include multi-antigen-binding compositions. In certain embodiments, the composition includes two or more antigen-binding components coupled to an elastin-like-polypeptide (ELP) scaffold utilizing two or more specific orthogonal protein interaction pairs. In certain embodiments, the specific orthogonal protein interaction pairs are two or more of SpyTag/SpyCatcher, SpyTag/KTag, Isopeptag/pilin-C, SnoopTag/SnoopCatcher, SpyTag002/SpyCatcher002, cohesin/dockerin, DogTag/DogCatcher, and SdyTag/SdyCatcher.

In certain embodiments, the multi-antigen-binding composition is used for detection of a biomarker indicative of an infectious disease, an autoimmune disease, an inflammatory disease, a metabolic disorder, a cardiovascular disease, a fibrotic disease, a neurological disorder, or a cancer. In certain embodiments, the biomarker is one or more of HER2, KRAS, EGFR, BRAF, BRCA1/BRCA2, prostate-specific antigen, carcinoembryonic antigen, and alpha-fetoprotein. In certain embodiments, a first antigen-binding component and a second antigen-binding component of the multi-antigen-binding composition are directed to detection of two of HER2, KRAS, EGFR, BRAF, and BRCA1/BRCA2. In some embodiments, the biomarker is one or more of C-reactive protein, procalcitonin, serum amyloid A, cytokines (IL-1, IL-6, TNF-α, calprotectin, ferritin, fibrinogen, cardiac troponins, BNP/NT-proBNP, and Lipoprotein(a), CK-MB and microRNAs.

In certain embodiments of the multi-antigen-binding composition, a first antigen-binding component binds to a tumor-specific antigen and a second antigen-binding component binds to T cell surface protein. In certain embodiments, the second antigen-binding component is anti-CD3. In certain embodiments of the multi-antigen-binding composition, a first antigen-binding component is anti-EGFR and a second antigen-binding component is anti-HER2. In certain embodiments of the multi-antigen-binding composition, a first antigen-binding component is anti-EGFR, a second antigen-binding component is anti-HER2, and a third antigen-binding component is anti-CD3.

Embodiments include a modular post-translational platform with highly specific protein-protein interactions for multi-functional antibody assembly and Elastin-like-polypeptide (ELP). Embodiments include methods of making these multi-functional antibodies that include the SpyCatcher002, SnoopCatcher, and dockerin fused with biocompatible ELP.

Provided herein are a modular post-translational platform with highly specific protein-protein interactions for multi-functional antibody assembly and Elastin-like-polypeptide (ELP) for easy purification. Multi-functional antibodies were generated and purified with almost 100% assembly efficiency and purity. Additionally, antibodies were assembled with diverse applications including detecting cancer, inhibiting cancer cell growth, and directing T cells to cancer cells for enhanced therapeutic efficacy. This platform offers high assembly efficiency, easy purification, and modularity for the redesign of antibody functions.

Embodiments of the method of making multi-functional antibodies are provided here. A novel protein scaffold method was developed for efficiently assembling multi-functional antibodies utilizing two or more highly specific orthogonal protein interaction pairs. In certain embodiments, three or more highly specific orthogonal protein interaction pairs are used. Examples of these protein interaction pairs include SpyTag/SpyCatcher, SpyTag/KTag, Isopeptag/pilin-C, SnoopTag/SnoopCatcher, SpyTag002/SpyCatcher002, cohesin/dockerin, DogTag/DogCatcher, and SdyTag/SdyCatcher.

One of the widely used protein-protein interactions is the SpyTag-SpyCatcher interaction. Originated from the bacterium Streptococcus pyogenes fibronectin-binding protein FbaB, the SpyTag/SpyCatcher binding pair is a protein ligation system based on the internal isopeptide bond of the CnaB2 domain of FbaB (see, e.g., Zakeri et al., Proc. Natl. Acad. Sci. USA. 2012; 109:E690-E697). CnaB2 is split and engineered into two complementary fragments, such that the first fragment (SpyCatcher) is able to bind and form a covalent isopeptide bond with the second fragment (SpyTag). SpyTag and SpyCatcher spontaneously and irreversibly form a covalent bond between lysine on SpyCatcher and aspartic acid on SpyTag when mixed. This reaction can tolerate a wide range of pH and temperature conditions, and both SpyTag and SpyCatcher can be fused to either N- or C-terminal of the protein of interest. Following the initial engineering of the SpyTag/SpyCatcher pair, SpyTag002/SpyCatcher002 was developed through phase-display library screening leading to an order of magnitude enhancement in interaction kinetics. Besides faster kinetics, orthogonal interaction pair SnoopTag/SnoopCatcher was also developed to form covalent bond between lysine on SnoopTag and Asparagine on SnoopCatcher by engineering the adhesin RrgA from Streptococcus pneumoniae.The SpyTag002/SpyCatcher002 and SnoopTag/SnoopCatcher protein pairs were used to facilitate the spontaneous covalent attachment between protein partners. Besides covalent bonding, the cohesin-dockerin pair was also used as this pair is one of the strongest known binding pairs from bacterial cellulosomes that helps multiple enzymatic subunits dock onto scaffolding subunits. Embodiments provided here include the SpyCatcher002, SnoopCatcher, and dockerin fused with biocompatible ELP, which allows easy separation of the target protein from host cell lysis through thermal precipitation cycles.

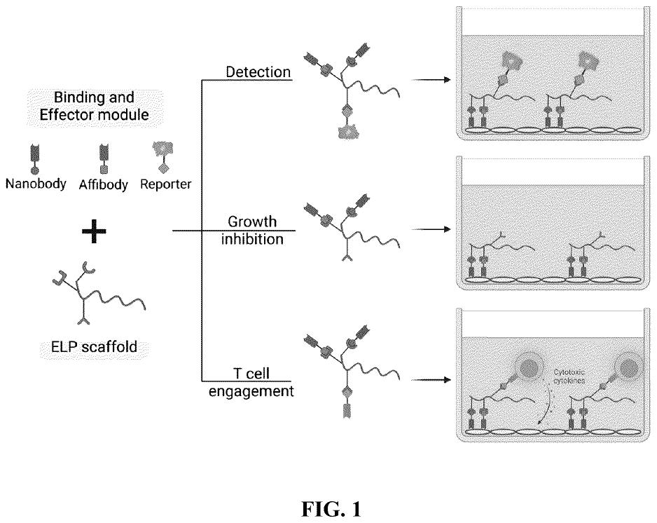

These multi-functional antibody compositions were assembled using nanobodies and/or affibodies. Nanobodies and affibodies are small biomolecules derived from the heavy-chain of camelidae family and staphylococcal protein A respectively. They provide an advantage for targeting biomarkers due to their low immunogenicity and high targeting specificity. This protein scaffold-based strategy achieves nearly 100% assembly efficiency by utilizing extremely precise protein-protein interactions. The purification challenge faced by multi-functional antibodies was addressed by inclusion of the ELP purification tag. The modularity of multi-functional antibodies for various applications were demonstrated by detecting cancer, inhibiting cancer cell growth, and directing T cells to cancer cells for high therapeutic efficacy (FIG. 1).

Embodiments can also include compositions that target one or more of any validated protein targets identified for therapy, such as HER2 (ERBB2) for breast, gastric, and ovarian cancers, KRAS for colorectal, pancreatic, and lung adenocarcinoma, EGFR for lung cancer, BRAF for melanoma and colorectal cancer, and BRCA1/BRCA2 for breast and ovarian cancer. Embodiments can also include compositions that target one or more of any validated biomarkers for screening or monitoring, such as the prostate-specific antigen for prostate cancer, carcinoembryonic antigen for colorectal and other gastrointestinal cancers, CA-125 for ovarian cancer, alpha-fetoprotein for liver cancer, and CA 19-9 for pancreatic cancer.

In an embodiment of the antibody assembly platform, HER2 and EGFR were chosen as the targeting cancer biomarkers, given their documented overexpression in various tumor types, including breast and lung cancer. Both HER2 and EGFR are part of the epidermal growth factor receptor family, and they are responsible for activation of the downstream signaling pathways that regulate cell cycle progression. Previous studies have shown that simultaneous inhibition of both HER2 and EGFR significantly enhances therapeutic efficacy.

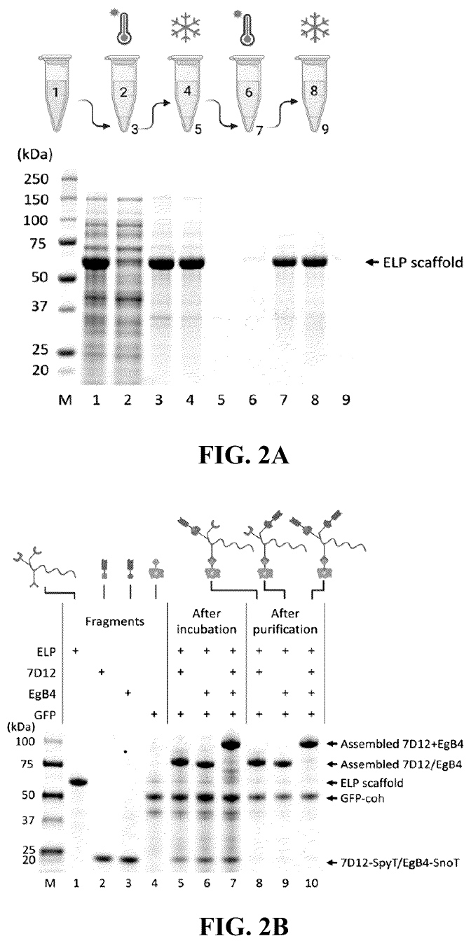

The ELP-SpyCather002-SnoopCather-Dockerin scaffold was expressed in BL21(DE3) cells, purified via two cycles of thermal precipitation with 1M (NH₄ )₂SO₄, and re-solubilization in ice-cold PBS (FIG. 2A). An antibody was first assembled with 7D12 and EgB4, which target different regions of EGFR. Nanobodies 7D12 and EgB4, fused with SpyTag002 and SnoopTag, respectively, were expressed in E. coli Shuffle T7, ensuring correct disulfide bond formation. Nanobodies fused with SpyTag002/SnoopTag were purified by Strep-Tactin columns through the C-terminal Twin-Strep-Tags. GFP was used as a fluorescent reporter. GFPcohesin fusion (GFP-coh) was expressed in BL21(DE3) cells. GFP-coh was purified by nickel-nitriolotriacetic acid (Ni-NTA) columns through the C-terminal His-tag.

For antibody assembly, an excess (1.5:1 molar ratio) of nanobody fusions and GFP-coh were incubated with the ELP scaffold. After incubation, the assembled antibody was purified by one round of thermal precipitation and cold re-solubilization to eliminate unbound nanobodies and GFP-coh. The purified antibody was analyzed by SDS-PAGE (FIG. 2B). Nearly 100% of the ELP scaffold was assembled with the nanobodies and GFP. Furthermore, the assembled antibodies were recovered after one cycle of thermal precipitation and cold re-solubilization resulting in >98% purity (FIG. 2B). As the cohesin-dockerin interaction is non-covalent, GFP-coh appeared as a separate band on SDS-PAGE (FIG. 2B).

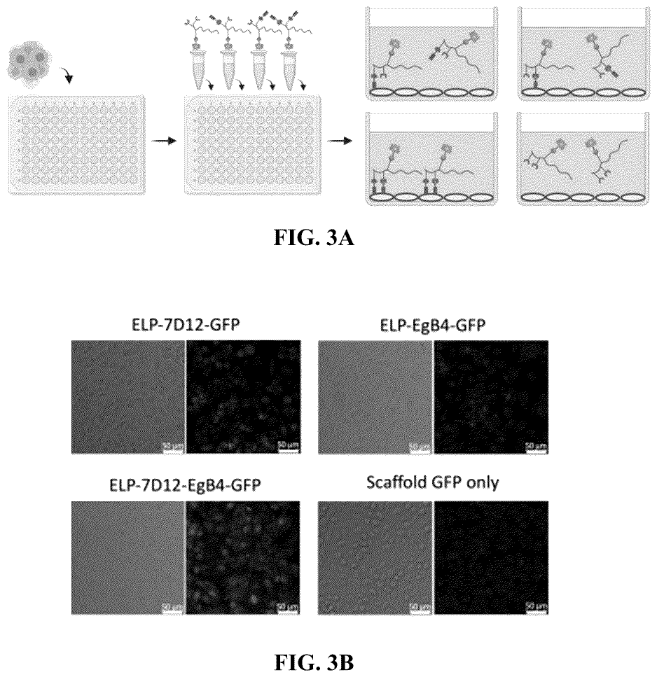

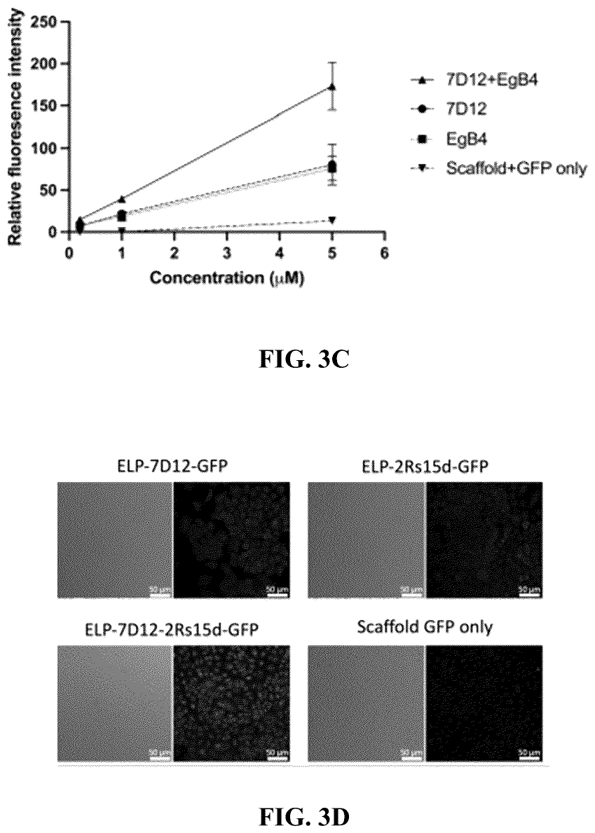

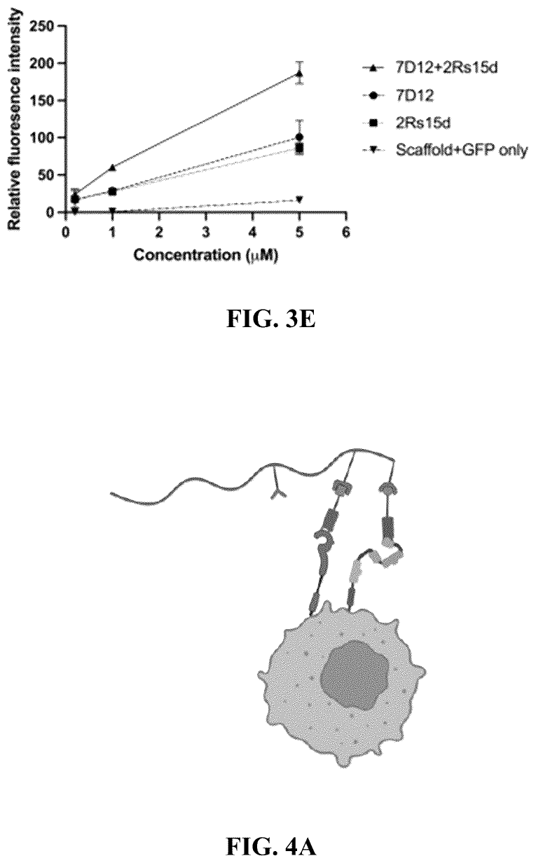

After antibody assembly, cancer detection was tested using MDA-MB-231, an epithelial human breast cancer cell line that overexpresses EGFR. The multivalence binding using the multi-functional antibody enabled a higher binding efficiency (FIG. 3A). The assembled antibody with single nanobody 7D12 or EgB4, which binds onto different regions of EGFR, and both nanobodies were tested by incubating with MDA-MB-231. The scaffold with only GFP-coh was included as a negative control. As shown from the image results, fluorescence intensity from the cells with assembled 7D12 and EgB4 showed two times higher fluorescence than with each nanobody alone. The scaffold without nanobody only showed little fluorescence background (FIGS. 3B, 3C). To showcase the modularity of this assembly method, we prepared another antibody combination featuring a 7D12 nanobody targeting EGFR and a 2Rs15d nanobody targeting HER2 using the same procedure. The assembled antibody was then incubated with A549 lung carcinoma epithelial cells that overexpress both EGFR and HER2 antigens. Similarly, the highest fluorescence intensity was observed from the antibody with both 7D12 and 2Rs15d (FIGS. 3D, 3E). This assembly method resulted in an efficient, precise, easy-purifiable, and modular antibodies with nanobodies and reporter fluorescent protein for cancer cell detection.

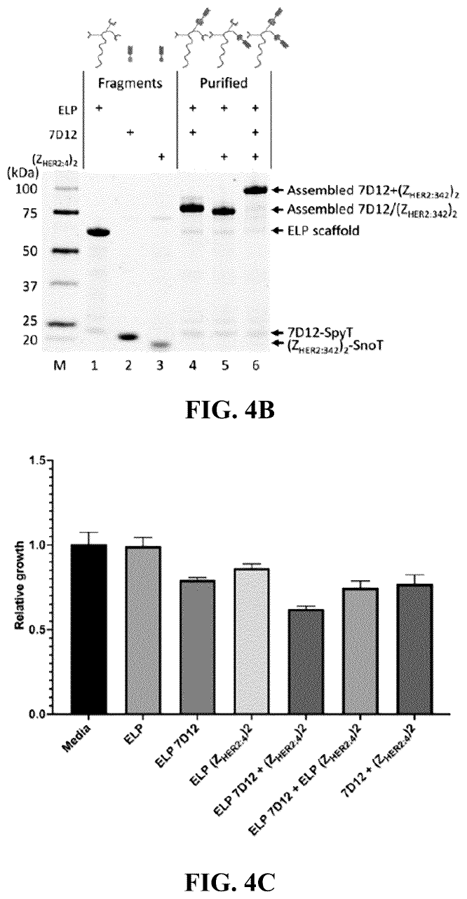

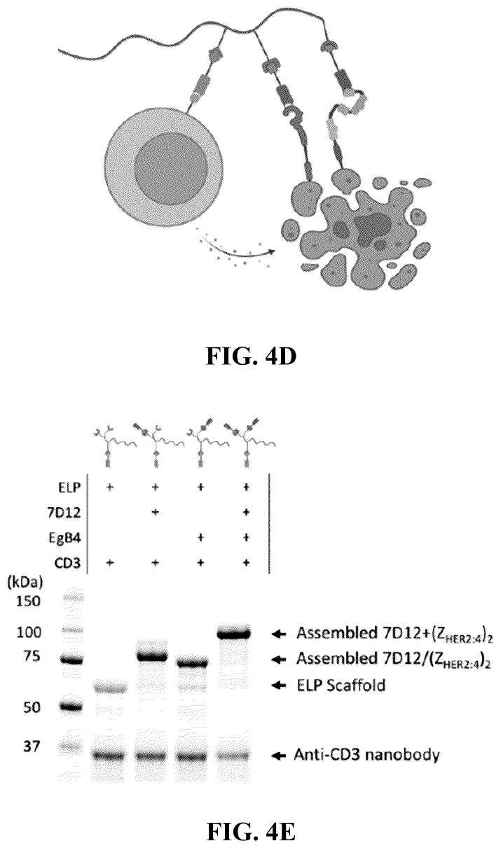

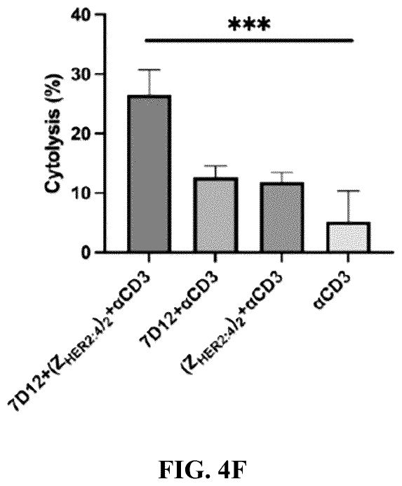

Besides detection, the assembled antibodies were tested for their ability to inhibit cancer cell growth (FIG. 4A). Nanobody 7D12 has been shown to inhibit cancer cell growth through binding with EGFR. Besides nanobodies, another embodiment constituting an affibody (Z_{HER2:4})₂ that targets HER2 was utilized. Similar to previous experiments for detection, nanobody 7D12 and affibody (Z_{HER2:4})₂ were assembled on the protein scaffolds, followed by one round of thermal precipitation and cold re-solubilization. SDS-PAGE analysis showed almost 100% assembly and purification efficiency (FIG. 4B).

Previous studies demonstrated that simultaneously targeting EGFR and HER2 would lead to superior therapeutic efficacy in suppressing tumor growth. But, there has been a lack of compositions that can achieve this goal. Embodiments of the assembled antibody provided here targeting both EGFR and HER2 antigens on A549 cells inhibited cancer cell growth more effectively. The protein scaffold did not show a significant inhibitory effect on the cell growth. The cells incubated with 7D12 antibody had a 15% decrease in the overall growth. Meanwhile, (Z_{HER2:4})₂ alone on the scaffold did not show a significant effect in inhibiting the cell growth, potentially because it was unable to inhibit HER2 phosphorylation. Surprisingly, the assembled antibody with both 7D12 and (Z_{HER2:4})₂ showed the most significant inhibition for cell growth (FIG. 4C). Compared to the cells grown in media, there was a 37% reduction in cell growth. Assembling both 7D12 and (Z_{HER2:4})₂ onto the ELP scaffold inhibited cell growth more effectively than having each nanobody or affibody alone.

To further improve the therapeutic efficacy and demonstrate the modularity of this method, an anti-CD3 nanobody was added onto the protein scaffold in addition to 7D12 and (Z_{HER2:4})₂. The effectiveness of anti-CD 3 nanobodies in both binding to and activating T-cells has been demonstrated. Using the embodiments herein, the multi-functional antibody compositions target EGFR and HER2 simultaneously to inhibit the cancer cell growth and recruits and activates T cells to mediate tumor lysis. Similar to previous experiments herein, 7D12, (Z_{HER2:4})₂, and anti-CD 3 nanobodies were assembled onto the scaffold. This assembly of the tri-functional composition was evaluated by SDS-PAGE. From the SDS-PAGE gel, almost 100% of the scaffold was assembled with the antibody fragments (FIG. 4E). After the assembly, each assembled antibody product was tested for their ability to induce cancer cell cytolysis against A549 cells. At the same effect-to-target ratio of 10:1 and a concentration of 1 μM for each assembled antibody, the antibody with 3 nanobodies targeting all CD3, HER2, and EGFR displayed the highest cytolysis (~27%). In contrast, the antibody with only anti-CD3 nanobody showed the least cytolysis (FIG. 4F). These results confirm that the assembled multi-functional antibody was able to recruit T cells to cancer cells and induce T cell mediated cytolysis against cancer cells. The modularity of the platform enables more possible antibody compositions for the flexibility of targeting different tumors and recruiting immune cells more efficiently.

There is an increasing interest in rapidly developing multi-functional antibodies. Embodiments provided here include a modular and easy-to-purify platform to assemble multi-functional antibodies for cancer detection and therapy with high efficacy. This highly specific and modular assembly approach provides the flexibility for combinations of antibody fragments, reporters, and immune cell engagers efficiently and cost-effectively. Using additional protein binding partners such as SdyTag/SdyCatcher (protein conjugation pair derived from the Streptococcus dysgalactiae FbaB protein) can potentially enable assembly of even more diverse antibodies.

ELP has enabled easy purification of the assembled antibody from whole cell lysis, eliminating the necessity of equipment/labor intensive purification. Furthermore, with ELP addition, antibody in vivo half-life can be longer than the non-IgG-like bi-functional antibodies because of the larger size. Using this post-translational assembly method, nanobodies, affibodies, and fluorescent proteins were assembled for detecting cancer, inhibiting cancer cell growth, and directing T cells to target cancer cells to further improve the therapeutic efficacy. This strategy can be used to assemble more antibody fragments using additional binding domains to enhance the binding efficiency and binding target diversity. Furthermore, the approach can be extended to simultaneously engage several T-cell recruiting and activation modules in one molecule for the best efficacy.

### EXAMPLES

Various examples are described to illustrate selected aspects of the various embodiments of multi-functional antibodies, including methods of making and use of these compositions.

Cloning and expression. ELP scaffold was constructed by first ligating Dockerin, SpyCatcher002, and SnoopCatcher gene fragments using Gibson assembly with (G₄S)₂ in between and then inserted into the pET24a-ELP-LPEGFT plasmid between BamHI and XhoI. (37) For each nanobody and affibody, their sequences were inserted into pET21a between XbaI and BamHI, a (G₄S)₂ linker and protein interaction pair (Coh, SpyT, SnoT) were inserted after the nanobodies between BamHI and EcoRI, and another (G₄S)₂ linker and Twin-strep tag were inserted at the C-terminal between EcoRI and XhoI. The ELP scaffold plasmid was transformed into BL21(DE3) for protein expression. Transformed bacteria were grown in Luria-Bertani (LB) medium supplemented with 50 μg/mL kanamycin, 1.5% glycerol, and 20 mM calcium chloride for proper dockerin folding. The culture was kept at 37° C. until OD600 reached 0.6, and then induced with 0.4 mM IPTG. The induced culture was kept at 30° C. overnight for protein expression. For the nanobodies and affibody expression, each plasmid was transformed into Shuffle T7 for proper disulfide bond formation. The bacteria were cultured in Terrific Broth (TB) at 37° C. until OD600 reached 0.6, and then induced with 0.4 mM IPTG. After induction, the cultures were moved to 30° C. overnight for protein expression.

Protein purification. ELP scaffold was purified by thermal precipitation and cold re-solubilization. After the overnight culture for protein expression, the cells were harvested and sonicated to obtain the cell lysate. The cell lysate was then added with (NH₄)₂SO₄ to a final concentration of 1 M, then incubated at 37° C. until the lysate turned cloudy. The lysate was then centrifuged at 37° C. and 20,000 g for 15 minutes. Supernatant was removed to remove the soluble impurities, and the pellet was resolubilized in cold PBS. After re-solubilization, the solution was again centrifuged at 4° C. and 20,000 g for 15 minutes. Supernatant was transferred to a new tube, and pellet that contained insoluble impurities was removed. This cycle was repeated to further improve the purity of the final product. For the nanobodies and affibody, as they contain a Twin-strep tag at C-terminal, they were purified through the Strep-Tactin column (IBA-lifesciences) following the manufacturer's instruction. Briefly, the column was first equilibrated 2 column volumes (CV) of wash buffer (100 mM pH 8.0 Tris/HCl, 150 mM NaCl, and 1 mM EDTA). Then the cell lysate was loaded onto the column. After that, the column was washed with 5 CV of wash buffer to remove unpacifically bound proteins. Then the protein was eluted by 3 CV the elution buffer (100 mM pH 8.0 Tris/HCl, 150 mM NaCl, 1 mM EDTA, 50 mM biotin). The column was then regenerated by 6 CV of regeneration buffer (3 M MgCl₂) followed by equilibration again with 8 CV of wash buffer.

Antibody assembly. Multi-functional antibodies were assembled by saturating the ELP scaffold to ensure complete binding of antibody fragments. Prior to assembly, the concentrations of antibody fragments and the ELP scaffold were quantified using a Nanodrop spectrophotometer. For the assembly, nanobodies or affibodies were mixed with the ELP scaffold at a molar ratio of 1.5:1. When additional fragments were required, all components were mixed together in a one-pot mixing step, with each antibody fragment combined with the ELP scaffold at a consistent 1.5:1 molar ratio. The mixture was incubated on a shaking platform at room temperature (60 rpm) for 4 hours. After incubation, a 2 μL sample was collected for SDS analysis to monitor assembly progress. Following incubation, the assembled antibodies underwent an additional purification step, involving one round of thermal precipitation followed by cold re-solubilization to remove any unbound nanobodies or affibodies. Purified products were then analyzed by SDS to confirm the removal of free antibody fragments. Assembled products were stored at −20° C. until use.

Cancer cell detection. MDA-MB-231 (EGFR+) and A549 (HER2+, EGFR+) cells were obtained from American Type Culture Collection (ATCC). MDA-MB-231 cells were cultured at 37° C. in non-CO₂ humidified incubator with Leibovitz's L-15 medium containing 10 vol % FBS and 1 vol% Pen/Strep. A549 cell were cultured at 37° C. in a 5% CO₂ humidified incubator with F12-K medium supplemented with 10 vol % FBS and 1 vol % Pen/Strep. For in vitro cell surface ligand binding assay, cells were seeded into black-wall, transparent-bottom 96-well plates and cultured at 37° C. until reaching around 90% confluency. Once the cells reached around 90% confluency, the growth media was removed, and cells were washed twice with PBST (1×PBS with 0.05% Tween 20). Cells were then fixed with 100 μL of 4% formaldehyde for 15 min and washed three times with 0.05% PBST. To minimize background adsorption, cells were incubated with 5% BSA for 15 minutes to block unspecific binding and washed three times with 0.05% PBS. Assembled antibodies were added to cells in specified concentrations for binding at 25° C. After one hour of incubation on a horizontal shaker, cells were washed three times with PBST to remove unbound antibodies before measuring the GFP fluorescence in 0.05% PBST with a plate reader. Cell without antibodies were also measured and recorded as background. Cells incubated with 5 μM of the assembled antibodies were also imaged using a Leica DMi8 fluorescence microscope.

Cancer cell growth inhibition. Therapeutic efficacy of the assembled antibodies was evaluated by cell growth inhibition assay using MTT (ThermoFisher). Cells were seeded in 96 well plates and allowed to grow at 37° C. overnight. Assembled antibodies were then added to the cells at a concentration of 5 μM and were incubated with the cells for additional 72 hours. After the incubation, media in the wells were replaced with 12 mM MTT dissolved in PBS and incubated for another 4 hours. MTT crystals formed at the end of the 4-hour incubation were dissolved in DMSO, and absorbance was measured at 540 nm using plate reader. Relative cell growth was calculated as Relative growth=(Absₛₐₘₚₗₑ−Abs_{10%DMSO})/(Abs_{media}−Abs_{10%DMSO}).

T cell mediated cytolysis. For T cell mediated cytolysis, A549 cancer cells and CD3+ T cells were mixed at a 10:1 ratio in each well of the 96-well plates. Each of the assembled antibody product was added into the wells at a concentration of 1 μM. After addition of the antibodies, the cells were then incubated at 37° C. in a 5% CO₂ humidified incubator for 48 hours. T cell mediated cytolysis was measured by lactate dehydrogenase (LDH) assay kit (ThermoFisher) following manufacturer's instruction. Spontaneous LDH levels were determined by wells with only A549 cells and T cells but without any antibody treatment. Maximum LDH levels was measured by incubating A549 cells with lysis buffer for 45 minutes. Cytolysis percentage was then calculated by the formula: Cytolysis=(Absₛₐₘₚₗₑ−Absₛₚₒₙₜₐₙₑₒᵤₛ)/(Absₘₐₓᵢₘᵤₘ−Absₛₚₒₙₜₐₙₑₒᵤₛ)*100%.

Provided here are multi-antigen-binding compositions, each containing two or more antigen-binding components coupled by an elastin-like-polypeptide (ELP) scaffold. These multi-antigen-binding compositions can be used for detection of a disease or for therapeutic treatment of a disease. The disease can be an infectious disease, an autoimmune disease, an inflammatory disease, a metabolic disorder, a cardiovascular disease, a fibrotic disease, a neurological disorder, or a cancer.

Provided herein are embodiments of a modular post-translational platform to produce these multi-functional compositions for a variety of applications, including cancer/virus targeting and immune cell activation and recruitment. In certain embodiments, these compositions are achieved by assembling two or more of a nanobody, an affibody, an antibody, or an Fc domain via highly specific protein-protein interactions and using of elastin-like-polypeptide (ELP) for easy purification. Embodiments include multifunctional antibody compositions that are characterized by modularity, high efficacy, and easy purification. They exhibit enhanced binding affinity and specificity, which lower the risk for on-target off-tumor specificities. The multifunctional antibodies here were generated and purified with almost 100% assembly efficiency and purity. Additionally, antibodies were assembled for diverse applications including detecting cancer, inhibiting cancer cell growth, and directing T cells to cancer cells for enhanced therapeutic efficacy. These multi-functional compositions can be assembled for detection and therapeutic applications of several diseases, such as an infectious disease, an autoimmune disease, an inflammatory disease, a metabolic disorder, a cardiovascular disease, a fibrotic disease, and a neurological disorder. This platform offers high assembly efficiency, easy purification, and modularity for the redesign of antibody functions.

Other objects, features and advantages of the disclosure will become apparent from the foregoing drawings, detailed description, and examples. These drawings, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein. It should be understood that although the disclosure contains certain aspects, embodiments, and optional features, modification, improvement, or variation of such aspects, embodiments, and optional features can be resorted to by those skilled in the art, and that such modification, improvement, or variation is considered to be within the scope of this disclosure.

What is claimed is:

1. A multi-functional composition, the composition comprising an elastin-like-polypeptide (ELP) scaffold containing two or more antigen-binding components or immunomodulatory agents and two or more specific orthogonal protein interaction pairs.

2. The multi-functional composition of claim 1, wherein the specific orthogonal protein interaction pairs are two or more of SpyTag/SpyCatcher, SpyTag/KTag, Isopeptag/pilin-C, SnoopTag/SnoopCatcher, SpyTag002/SpyCatcher002, cohesin/dockerin, DogTag/DogCatcher, and SdyTag/SdyCatcher.

3. The multi-functional composition of claim 1 for use in detection of a biomarker indicative of an infectious disease, an autoimmune disease, an inflammatory disease, a metabolic disorder, a cardiovascular disease, a fibrotic disease, a neurological disorder, or a cancer.

4. The multi-functional composition of claim 3, further comprising a reporter.

5. The multi-functional composition of claim 4, wherein the reporter is a fluorescent reporter, a bioluminescent reporter, an enzymatic reporter, or an affinity tag.

6. The multi-functional composition of claim 3, wherein the biomarker is one or more of HER2 (Human Epidermal Growth Factor Receptor 2), KRAS (Kirsten Rat Sarcoma Viral Oncogene Homolog), EGFR (Epidermal Growth Factor Receptor), BRAF (B-Raf Proto-Oncogene, Serine/Threonine Kinase), BRCA1/BRCA2 (Breast Cancer Genes 1 or 2), prostate-specific antigen, carcinoembryonic antigen, and alpha-fetoprotein.

7. The multi-functional composition of claim 1 for use in treatment of an infectious disease, an autoimmune disease, an inflammatory disease, a metabolic disorder, a cardiovascular disease, a fibrotic disease, a neurological disorder, or a cancer.

8. The multi-functional composition of claim 7 for use in treatment of cancer.

9. The multi-functional composition of claim 8, wherein a first antigen-binding component and a second antigen-binding component are directed to two of HER2, KRAS, EGFR, BRAF, and BRCA1/BRCA2.

10. The multi-functional composition of claim 8, further comprising one tumor-specific antigen-binding component and one T cell surface protein-binding fragment.

11. The multi-functional composition of claim 10, wherein T cell receptor-binding fragment is anti-CD3.

12. A multi-antigen-binding composition, the composition comprising two or more antigen-binding components coupled to an elastin-like-polypeptide (ELP) scaffold utilizing two or more specific orthogonal protein interaction pairs.

13. The multi-antigen-binding composition of claim 12, wherein the specific orthogonal protein interaction pairs are two or more of SpyTag/SpyCatcher, SpyTag/KTag, Isopeptag/pilin-C, SnoopTag/SnoopCatcher, SpyTag002/SpyCatcher002, cohesin/dockerin, DogTag/DogCatcher, and SdyTag/SdyCatcher.

14. The multi-antigen-binding composition of claim 12 for use in detection of a biomarker indicative of an infectious disease, an autoimmune disease, an inflammatory disease, a metabolic disorder, a cardiovascular disease, a fibrotic disease, a neurological disorder, or a cancer.

15. The multi-antigen-binding composition of claim 14, wherein the biomarker is one or more of HER2, KRAS, EGFR, BRAF, BRCA1/BRCA2, prostate-specific antigen, carcinoembryonic antigen, and alpha-fetoprotein.

16. The multi-antigen-binding composition of claim 12, wherein a first antigen-binding component and a second antigen-binding component are directed to detection of two of HER2, KRAS, EGFR, BRAF, and BRCA1/BRCA2.

17. The multi-antigen-binding composition of claim 12, wherein a first antigen-binding component binds to a tumor-specific antigen and a second antigen-binding component binds to T cell surface protein.

18. The multi-antigen-binding composition of claim 17, wherein the second antigen-binding component is anti-CD3.

19. The multi-antigen-binding composition of claim 12, wherein a first antigen-binding component is anti-EGFR and a second antigen-binding component is anti-HER2.

20. The multi-antigen-binding composition of claim 12, wherein a first antigen-binding component is anti-EGFR, a second antigen-binding component is anti-HER2, and a third antigen-binding component is anti-CD3.
